(19) United States
(12) Patent Application Publication
(10) Pub. No.: US 2005/0043676 A1
(43) Pub. Date: Feb. 24, 2005

(54) Means for administering an injectable product in doses

(76) Inventors: Axel Remde, Luthelfluh (CH); Hanspeter Heiniger, Lotzwil (CH)

Correspondence Address: David E. Bruhn, Esq.;DORSEY & WHITNEY LLP, Intellectual Property Department, 50 South Sixth Street, Suite 1500, Minneapolis, MN, 55402-1498, US

(21) Appl. No.: 10/909,256

(22) Filed: Jul. 30, 2004

Related U.S. Application Data
(63) Continuation of application No. PCT/CH03/00040, filed on Jan. 21, 2003.

(30) Foreign Application Priority Data
Feb. 1, 2002 (DE) 102 04 119.9

(51) Int. Cl. A61M 31/00

(52) U.S. Cl. 604/67

(57) ABSTRACT

A device for dispensing precise amounts of a substance including at least one detector coupled to the device for detecting accelerations, wherein the detector device is triggered and/or read in a continuous or clocked manner and reversibly or irreversibly changes an indicator when a selected acceleration threshold is exceeded.

### CROSS-REFERENCE TO RELATED APPLICATION(S)

This application is a continuation of and claims priority to International Application No. PCT/CH03/00040, filed on Jan. 21, 2003, which claims priority to German Application No. 102 04 119.9, filed on Feb. 1, 2002, the contents of both applications are incorporated herein by reference in their entirety.

### BACKGROUND

The present invention relates to devices, apparatus and methods for administering or dispensing substances, for example injectable products, in selected or measured amounts or doses. More particularly, the present invention relates to means for the long-term administering of doses of a medicinal agent, for example a medically active agent such as insulin, etc. The means for administering or dispensing referred to and/or described herein are intended to encompass suitable devices, apparatus, machines, articles of manufacture, structures, processes, etc. In one embodiment, the means take the form of a portable infusion pump.

Such means for administering, as known from the prior art, principally comprise a container for storing the injectable product, for example a liquid containing a medical active agent, and an administering mechanism for administering the product in doses. While injection means, such as those for administering insulin, predominantly used to be configured for disposable use and the patient was thus accorded a large degree of personal responsibility for administering a correct dosage, such injection means are increasingly configured with a reservoir, for example an ampoule, for administering a number of individual doses and with a simple and reliable dosing mechanism which is intended to exclude or minimize the danger of incorrect dosing as far as possible. An example of such means is known from DE 199 00 827 C1, belonging to the Applicant hereof. For long-term therapy, portable infusion pumps are known to comprise an autonomous energy supply and an electronic control system which suitably controls a micro-pump in order to administer suitable micro-doses of a medical active agent over an extended period of time.

In such means, the danger of incorrect dosing must be eliminated or reduced as far as possible. Since such means for autonomous self-medication are formed to be increasingly transportable and/or portable, mechanical influences on the means, e.g., jolts, in particular represent an increasingly significant cause of incorrect dosing. The patient cannot, however, always be sure of a correct dosage, despite the means appearing externally to be intact. Excessively high accelerations, for example, can lead to mechanical damage to the ampoule accommodating the product or to mechanical parts of the apparatus or device. Liquid penetrating into the means can cause the mechanical parts—for example, in a micro-pump—to slip, such that despite the operability of the apparatus, a required dosage is not administered. Extreme mechanical stresses can occur both within the patient's sphere of influence, for example when carrying the means around, or also by way of the distribution or shipping path from the manufacturer to the patient.

Due to the respective legal situation, manufacturers of such means increasingly have to handle warranty claims. Simple measures are therefore desirable, in order to be able to determine whether extreme mechanical stresses have occurred within the patient's sphere of influence or elsewhere, for example, during distribution.

Jolt indicators are known from the prior art which are added to freight consignments and experience an irreversible coloration if mechanical stresses occur in transit which exceed a threshold value.

### SUMMARY

It is an object of the present invention to provide a device or apparatus, i.e., means, for administering an injectable product in doses in which the danger of incorrect dosing due to mechanical stresses is reduced. In one embodiment, the device or apparatus comprises a portable infusion pump adapted for the long-term administering of a medically active agent.

This object is addressed by providing means for administering an injectable product in doses, in one embodiment comprising a container for storing the injectable product and an administering mechanism for administering the product in doses, comprising at least one detector means for detecting accelerations, wherein each detector means is rigidly connected to a casing section of the means for administering.

In one embodiment, the present invention comprises a device for dispensing precise amounts of a substance comprising at least one detector operably coupled to the device and at least one indicator operably coupled to the device and to the detector, said detector for detecting accelerations, wherein the detector is at least one of triggered and read in one of a continuous or clocked manner and prompts the indicator to change when a selected acceleration is detected. The change may be either reversible or irreversible.

In one embodiment, the present invention comprises a device for dispensing precise amounts of a substance including at least one detector coupled to the device for detecting accelerations, wherein the detector device can be triggered and/or read in a continuous or clocked manner and can reversibly or irreversibly change an indicator when a selected acceleration threshold is exceeded. The acceleration thresholds are established on the basis of empirically or theoretically determined thresholds.

In accordance with the invention, a means for administering an injectable product in doses comprises at least a detector means for detecting accelerations, wherein the detector means is rigidly connected to a casing section of the infusion means. The invention is thus based on the recognition that extreme mechanical stresses, for example knocks to a casing, shaking during transport or handling, etc., result in corresponding accelerations occurring. Since acceleration recorders are available cost-effectively and in a large number of embodiments, extreme mechanical stresses can be cost-effectively and simply demonstrated in accordance with the invention.

Advantageously, the accelerations occurring are substantially proportional to the intensity of the mechanical stresses occurring. Thus, in accordance with the invention, a simple measurement variable is provided which can be simply used to make a statement of the danger of the means incorrectly dosing. For, aware of the specific configuration of the infusion and/or injection means, for example the nature and strength of the attachments, the material properties of essential elements of the means such as for example the ampoule or the pump, etc., or on the basis of experimental values which can be obtained from standard tests such as drop tests etc., a quantitative statement of the danger of incorrect dosing can be simply made from the magnitude of a detected acceleration. If, for the reasons cited above by way of example, it is known for the means that when a limiting acceleration value is exceeded, the probability of apparatus errors escalates, then suitable measures are expediently taken to indicate that the limiting acceleration value has been exceeded.

In accordance with one aspect of the present invention, a state of a detector means changes irreversibly when a detected acceleration exceeds a predetermined threshold value. This change in state can be indicated to the user on a display means or can be electronically detected and further processed. In accordance with a preferred embodiment, the detector means is a commercially available jolt indicator which, in the prior art, is added to consignments of goods and experiences a change in color when a predetermined acceleration limit value is exceeded. Such a jolt indicator can, for example, be formed as a tube or capillary which is partly filled with a drop of dye held together by its defined surface tension. If the accelerations occurring overcome the surface tension, the dye is distributed in the whole capillary, leading to an irreversible coloration of the capillary which can easily be detected or read.

In accordance with one embodiment of the present invention, a glass capillary is exchangeably inserted into a viewing window of the casing of the means (means referring to a administering, delivery or dispensing device or apparatus in accordance with the present invention), such that the glass capillary is visible from the outer side or outside of the casing. Advantageously, the user can immediately recognize a change in color and decide whether to return the means to the manufacturer or have it checked. A retailer, when unpacking the means, can also simply determine whether extreme mechanical stresses have occurred during transport which would make it seem advisable to return the means to the manufacturer.

In accordance with another preferred embodiment, the detector means is read electronically and the signals received are electronically processed further. For example, it is possible to provide for a warning indication to be indicated on a display when it is determined that an acceleration limit value has been exceeded. A block or lock, which disables all or a part of the device, can also be triggered as applicable, which however is often not expedient while the means is being operated, since it may be a medically active agent or the like which is to be administered. Therefore, means in accordance with such an embodiment is preferably coupled to a means for deciding whether the means has already been operated for the first time or not, wherein the block is only triggered if the means has not yet been operated for the first time. This can, for example, be determined on the basis of a pin code, a start procedure for operating the means, activation of a main energy source, etc.

The detector means can be read by a light detector array which detects a change in color, clouding or the like in a glass capillary of the aforesaid or another type. For this purpose, the light detector array expediently comprises a light-emitting element and, on the opposite side of the glass capillary, a light-receiving element. A control circuit controls the light detector array and determines, on the basis of the signal output by the light-receiving element, whether a change in color, clouding, etc. indicating an extreme mechanical stress has occurred.

In accordance with another embodiment, the detector means can also be read electrically. A thin wire can, for example, be provided in a capillary, wherein the wire tears when a predetermined acceleration limit value is exceeded and thus causes a change in resistance. This can be detected by conventional measurement circuits, for example a Wheatstone bridge circuit, which is read, continuously or clocked, by a control and/or measurement circuit. In addition or as an alternative to the thin wire, a liquid can also be provided in a capillary, wherein a change in resistance occurs between two measurement electrodes, preferably provided on the facing sides of the capillary, when the detected acceleration exceeds a predetermined or selected acceleration limit value. For this purpose, a membrane can, for example, be provided in a capillary, which separates two liquids of different electrical conductivity from each other and tears when the acceleration limit value is exceeded. The function of the membrane can in principle also be replaced by a surface tension of a liquid, as described above.

In accordance with a preferred embodiment, directed in particular to a portable infusion pump, the detector means is read synchronously with the administration or delivery of the product to be injected, which can be periodic or non-periodic. Advantageously, energy consumption can be reduced even further, since the electronic control and/or reading system is only activated when product is being delivered. In the remaining time intervals, the means can be switched off or operated in a standby mode with low energy consumption.

When the detector means is read, continuously or clocked, a storage means can also be provided for storing a time data value which indicates at which point in time or in which time interval the detector means irreversibly changed its state. A simple, quantitative statement can thus be made not only with regard to the danger of an incorrect diagnosis but also with respect to the fault (with the manufacturer, during distribution or with the user).

In accordance with another embodiment, the detector means can also change a state when a detected acceleration exceeds the predetermined acceleration limit value. Advantageously, even a number of mechanical extreme stresses can be reliably detected and recorded in this simple way. In accordance with this embodiment, the detector means is preferably formed as an acceleration switch which reversibly changes a switching state when the detected acceleration exceeds an acceleration limit value. Such a change in the switching state can be simply read using conventional measurement circuits.

In principle, however, the detector means can also be configured such that the accelerations occurring are continuously detected, for example by means of electronic sensors. In order to save on energy, an evaluation circuit is preferably provided with a comparator means, in order to compare the detected acceleration with a threshold value which can be predetermined, wherein signals are only forwarded to an evaluation unit when the threshold value is exceeded.

In embodiments in which the detector means is read electronically, for example with detectors which reversibly change their state or with electronic sensors, a storage means is preferably provided for storing a number of discrete data sets which each indicate that the detected acceleration in each case has exceeded the predetermined threshold value. The data sets can expediently comprise other information, for example, a time value for when the detected acceleration exceeds the predetermined threshold value, a time period during which the detected acceleration exceeds the predetermined threshold value, the value of the detected acceleration and/or other conditional or operational parameters.

To further reduce data—in particular when using continuously detecting sensors—the storage means can be operated in the manner of a FIFO memory, wherein the data sets are ordered according to the value of the detected acceleration, and the data set with the smallest value for the detected acceleration in each case is replaced by the data set with the next largest acceleration when the value for the detected acceleration is larger than the acceleration of the data set with the smallest value for the detected acceleration in each case, and wherein a data set with the currently detected acceleration is additionally stored in the storage means.

For a simple quantitative statement of the magnitude of the mechanical stresses occurring, it can be sufficient for the detector means to respond isotropically, i.e., not direction-dependently, to accelerations. In accordance with a preferred embodiment, however, the detector means responds direction-dependently to accelerations, which can offer other advantages when evaluating. For example, aware of the position of the means in a transport package, a possible cause of the mechanical stress having occurred can be identified. Or, aware of the position and orientation of the components of the means critical to failure, a more exact statement of the danger of incorrect dosing can be made. This can be significant when processing liability and warranty claims.

In accordance with a preferred embodiment, two or three detector means are provided which respond to accelerations along respective mutually orthogonal directions, such that the accelerations having occurred can be indicated, direction-resolved, in a plane or in three-dimensional space. The detector means are particularly expediently orientated parallel to a printed circuit board in the means, which, for example, has, bears, carries or is coupled to a control circuit for the means for administering, e.g., a portable infusion device, such that the danger of the electronic control system failing can also be effectively quantitatively detected.

Other features and advantages of methods and apparatus of the present invention will become more fully apparent and understood with reference to the accompanying description, drawings and claims.

### BRIEF DESCRIPTION OF THE DRAWINGS

FIG. 1 shows an example of a jolt indicator in a normal state (A) and in a state after a change in color (B) which occurs when an acceleration occurs which exceeds a threshold value;

FIG. 2 schematically shows components of one embodiment of means in accordance with the present invention, which offers the option of detecting an event once and storing the point in time of the event;

FIG. 3 schematically shows parts of means in accordance with another embodiment of the present invention, having the option of repeatedly detecting events and storing the points in time of the events;

FIG. 4 schematically shows an acceleration-sensitive switch which responds to accelerations in one direction;

FIG. 5 schematically shows—in two cross-sectional views—the arrangement of acceleration-sensitive switches in accordance with FIG. 4, in a means in accordance with another embodiment of the present invention;

FIG. 6 schematically shows the arrangement of acceleration-sensitive switches in accordance with FIG. 4, in another embodiment in accordance with the present invention; and

FIG. 7 schematically shows parts of an embodiment of the present invention, including an electronic evaluation and control system.

### DETAILED DESCRIPTION

In the figures, identical reference numerals designate identical or functionally identical elements, sub-assemblies or features.

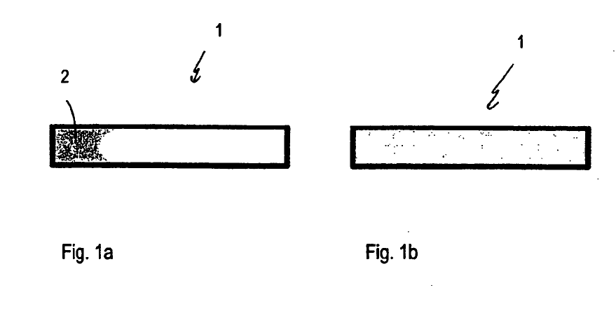

FIG. 1 shows a shock or jolt indicator for product monitoring during transport. This type of indicator is, for example, available from the firm Stroebel under the designation Shockwatch®. In accordance with the present invention, such a shock or jolt indicator can be used to detect an acceleration once. FIG. 1a shows the jolt indicator in an initial state. The jolt indicator comprises a glass capillary 1 (which could be formed of material other than glass). In the left-hand part of the glass capillary, a drop of dye 2 is inserted, the surface of which is curved into the meniscus shown due to the effects of surface tension. The remaining inner space of the glass capillary is empty. The surface tension is dependent on the liquid and on the geometrical relationships as well as on the material of the capillary 1 and clearly defines an acceleration limit value. If this is exceeded, then the surface tension is overcome and the dye distributes itself in the whole capillary, which leads to an irreversible coloration, a change in color, a clouding or the like in the right-hand part of the capillary and can be detected by the human eye or by an opto-electronic detection means. The state of the glass capillary 1 after the change in color is shown in FIG. 1b, in which a colored film has distributed itself evenly onto the walls of the capillary.

A jolt indicator, such as that shown in FIG. 1 or another suitable type, can be visible from the outer side of a casing of an infusion and/or injection means in accordance with the present invention. For this purpose, a viewing opening can be provided in the casing of the means, or the jolt indicator can be arranged in the area of an ampoule of the means which can be viewed from without. If the jolt indicator is read opto-electronically, it can alternatively also be arranged on an inner side of the casing, not viewable from without. The glass capillary can of course be exchanged at any time, for example at the manufacturer, retailer or during service.

The jolt indicator shown is rigidly connected, indirectly or directly, to a casing section of the infusion means. If, for example, one wants to detect the mechanical stresses acting on an electronic control system provided in the means, then the jolt indicator is expediently mounted together with the electronic control system, for example on a printed circuit board or the like. If one is more interested in the mechanical stresses acting on the mechanical parts of the means, then the jolt indicator is mounted together with such mechanical parts at a suitable point, for example together with a micro-pump of a portable infusion means or an ampoule containing a medical active agent. Suitable methods and techniques of attachment will be clear to the person skilled in the art when studying this description.

The jolt indicator shown in FIG. 1 reacts substantially isotropically, i.e., independently of the direction of the acceleration vector. Jolt indicators which respond anisotropically are, however, also known. These can be expediently arranged in order to increase response reliability, for example along mutually orthogonal axes.

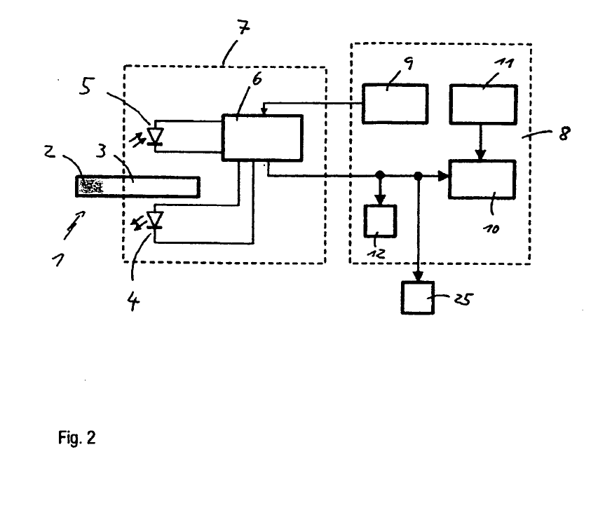

FIG. 2 shows parts of means in accordance with a first embodiment of the present invention having the option of detecting an event once and of storing the time of the event. The stored time information may include the time the event occurred, the duration of the event, etc. For this purpose, the glass capillary 1 in accordance with FIG. 1—shown in the left-hand edge of the image—is arranged in a light detector array 7 which comprises a light-emitting element 4, for example an LED, a light-receiving element 5, for example a photo-detector, and a control circuit 6 for controlling the LED 4 and the photo-detector 5. The control circuit 6 can be operated continuously or clocked in regular or non-regular intervals. When an acceleration limit value predetermined by the surface tension of the drop of dye 2 in the glass capillary 1 is exceeded, the change in color, clouding or other change in an optical parameter as described above occurs, which is detected by the light detector array 7 immediately or at the next measurement process. The control and evaluation circuit 6 can output the reading continuously. Preferably, however, the control and evaluation circuit 6 only changes an output signal after determining the change in color, clouding or other change in an optical parameter as detected by the photo-detector 5.

In order to control the control and evaluation circuit 6, it can be connected to timer 9 which emits a clock signal which triggers a reading process of the light detector array 7. The signal at the output of the control and evaluation circuit 6 can be used by a subsequent alarm means 12, a data memory 10 for storing event values and an external display means 25.

In accordance with a preferred embodiment, the array is not operated permanently, in order to minimize energy consumption, but merely cyclically polled, for which the clock signal of the timer 9 of the pump control system 8 is used. Once an acceleration has occurred which exceeds the surface tension of the drop of dye 2, the change in color or the like is detected during the next reading cycle. This event is stored in the data memory 10 of the means together with a time read from the system clock 11. Furthermore, an alarm system 12 can be activated, also an external display 25, for example for providing or indicating a warning indication for the user. The latter can then make a decision, on the basis of the display, as to whether the means is to be returned to the manufacturer or retailer, to be checked, repaired, etc. The means can also be automatically stopped, disabled or locked, though this is preferably not done, since it is preferably a medical active agent which is to be administered using the means. As a compromise solution, the pump control system 8 can detect whether the means has already been operated for the first time, and the pump is only stopped or blocked if the pump has not yet been operated for the first time.

Thus, in one embodiment, the state of the indicator can be cyclically polled and the occurrence of an event, together with the point in time of occurrence or time interval of occurrence, respectively, can be stored in the data memory of the pump control system 8. The data memory 10 is preferably a non-volatile memory, such that the event data cannot be lost.

The jolt indicator shown in FIG. 2 can, of course, also be viewable from the outer side of the casing of the means, such that the user can recognize extreme mechanical stresses even if the electronic control system means has not yet been operated, for example because a battery has not yet been inserted.

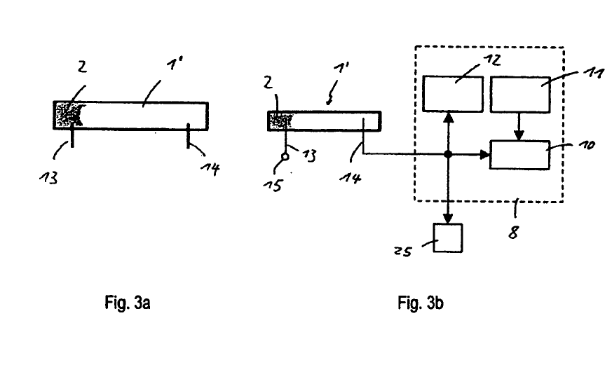

FIG. 3 shows an example of a detector means in accordance with the present invention, which can be read electronically. It is substantially formed as a tubular glass capillary 1′ and comprises, at least in its left-hand part, a colored or colorless drop of liquid 2, the surface of which is curved into the meniscus shown due to the effects of surface tension. When an acceleration limit value is exceeded, the meniscus tears and the liquid 2 pours out over the whole glass capillary 1′ or as applicable mixes with a liquid provided in the remaining area, said liquid exhibiting a different electrical conductivity. A first electrode 13 is fused into the glass capillary 1′ in the left-hand area of the glass capillary 1′ and a second electrode 14 is fused into the glass capillary 1′ in the right-hand area of the glass capillary 1′. As is shown, before the indicator responds, only the left-hand electrode 13 protrudes into the liquid 2. Once the indicator has responded, an electrical contact between the electrodes 13, 14—similar to a mercury switch—is caused or changed due to the electrical conductivity of the liquid 2 or of another liquid provided in the glass capillary 1′. The electrical resistance between the electrodes 13, 14 can be measured in a way known in the prior art, for example by means of a Wheatstone bridge circuit.

FIG. 3b shows another way of wiring the capillary 1′ in accordance with FIG. 3a. In this case, one electrode 13 lies on a fixed reference potential 15, while the other electrode 14 is connected directly to the pump control system 8, which by way of example, comprises a data memory 10 for storing event values, a system clock 11, an alarm means 12 and an external display 25.

Such a design offers at least three advantages:

the array operates without power before an event occurs and thus does not burden the energy source of the means. The other electrically operated elements provided in the means, for example a micro-pump, can thus be operated for longer. In order to further reduce energy consumption, the indicator can be separated off by a (semiconductor) switch—not shown in FIG. 3b—once an event has occurred, thus preventing a permanent power flow. For this purpose, a trigger switch or the like is provided which can detect a change in the resistance or potential in the means and convert this into a control signal;

a more space-saving design results as compared to the solution in accordance with FIG. 2, since the light detector array is omitted; and

an event is immediately registered as it occurs, thus enabling an immediate reaction by the user as applicable. This increases the operational reliability of the means still further, in particular when liquid directly enters the pump, for example through a fracture or crack in the ampoule containing the medical active agent.

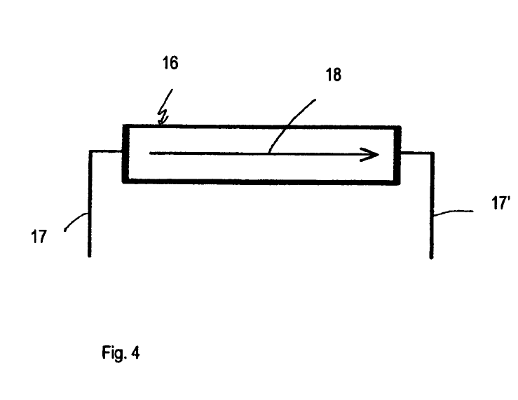

While the above embodiments have been directed to detecting an event once, events can also be repeatedly detected and stored in accordance with the present invention. For this purpose, a suitable acceleration recorder may be used which reversibly changes a state when a mechanical stress—as indicated by an acceleration occurring—occurs. An example of such an acceleration detector is acceleration-sensitive switches such as are tendered by the firm Assemtech of Great Britain. FIG. 4 schematically shows such an acceleration-sensitive switch 16 comprising two measurement contacts 17, 17′ and a measurement axis 18 which is predetermined by the casing of the switch 16 and indicates the direction of the accelerations which can be detected by the switch 16. The acceleration switch shown in FIG. 4 only closes when an acceleration occurs in the direction of the arrow which is above a response threshold of the switch 16. Such an acceleration-sensitive switch 16 thus usually reacts anisotropically and only in a direction along the measurement axis 18.

The acceleration-sensitive switch 16 in accordance with FIG. 4 can be installed in a casing of an injection or infusion means individually or together with a number of similar switches. Different types of such acceleration-sensitive switches, having different response thresholds, can also be used, such that stepped quantitative statements can be made with respect to the probability of errors in the means.

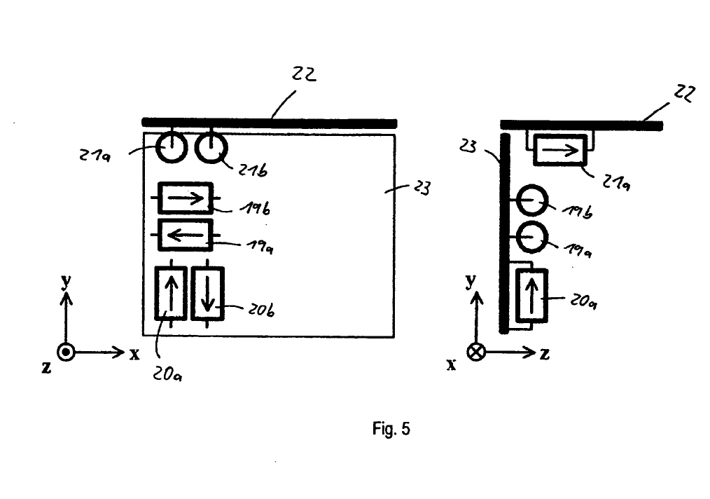

FIG. 5 shows, as an example of using a number of acceleration-sensitive switches in accordance with FIG. 4, parts of another embodiment in accordance with the present invention, having the option of repeatedly detecting events, direction-resolved, and storing the points in time of the events. Reference numerals 22 and 23 designate optional casing sections of an injection or infusion means or printed circuit boards or the like attached in such means for accommodating an electronic control system, etc. (not shown). As indicated by the two coordinate systems, the sections 22 and 23 are mutually orthogonal. A pair of acceleration-sensitive switches 21a, 21b are rigidly attached to the section 22 and respond to accelerations occurring in opposite directions. Two mutually orthogonal pairs of acceleration-sensitive switches 19a, 19b and 20a, 20b, respectively, are attached to the section 23 and each respond to accelerations occurring in opposite directions along the schematically indicated measurement axes. The acceleration switches shown can be read individually, such that a statement is also possible with respect to the direction of the acceleration having occurred. The acceleration switches shown can in principle also be connected in parallel or can be read in a multiplex operation.

The acceleration-sensitive switches shown in FIGS. 4 and 5 preferably operate in binary, i.e., such that a change in signal can be detected when a threshold value predetermined by the acceleration-sensitive switch being used in each case is exceeded. A statement beyond this, of the magnitude of the acceleration, is not possible in this embodiment, except for the option of making a statement of the direction in which the detected accelerations have (not) exceeded the threshold value.

Acceleration sensors are known to the person skilled in the art from the prior art which respond substantially linearly to accelerations occurring. Such acceleration sensors are based on very different measurement principles. For the purposes of the present invention, sensors are preferably used which can be electrically read, but other suitable sensors may be used as well or in addition. The measurement principles can for example utilize piezo-electrical, piezo-resistive or capacitive effects. Such acceleration sensors usually react anisotropically to accelerations occurring, for example along a preferential axis of the sensor.

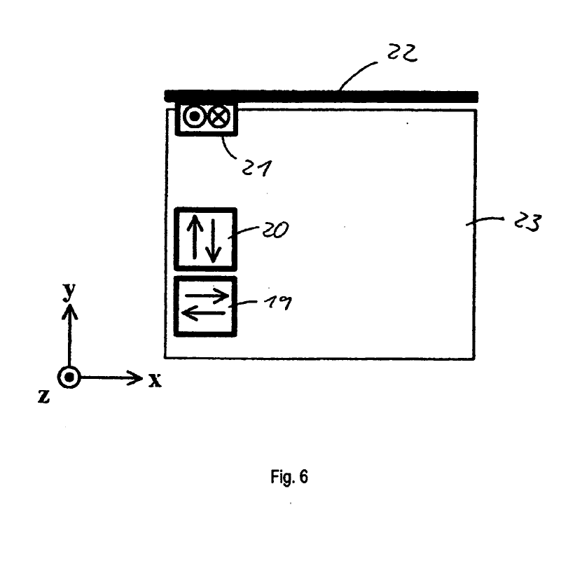

In accordance with the present invention, FIG. 6 shows an example of an embodiment having the option of repeatedly detecting events and storing the points in time of the events, as well as a magnitude of the acceleration having occurred. FIG. 6 shows a design comprising three uniaxial sensors 19, 20 and 21, which are attached to two casing sections and/or printed circuit boards 22, 23 in such a way that their measurement axes are mutually orthogonal. The acceleration sensors 19 to 21 can respond linearly or non-linearly to accelerations occurring. In order to relieve the electronic evaluation system—still to be described below—a threshold value function can also be provided, such that the acceleration sensors 19 to 21 only respond, preferably linearly, once the predetermined acceleration limit value has been exceeded.

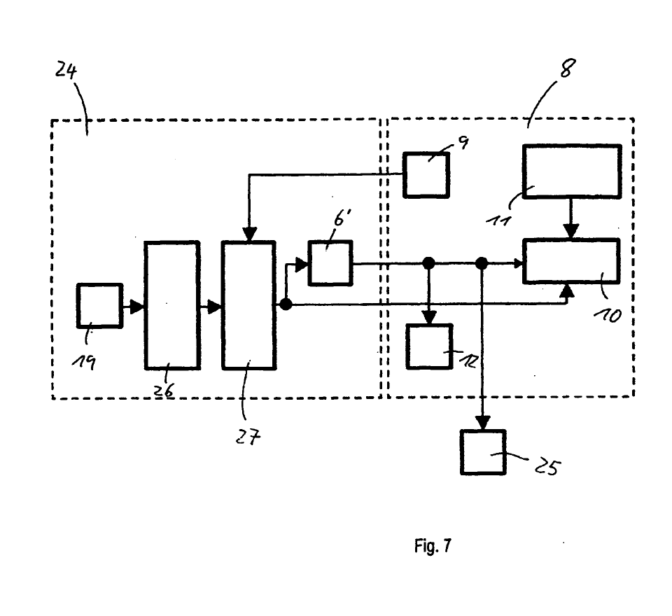

FIG. 7 schematically shows the signal processor for the output signals provided by the acceleration sensors 19 to 21 in accordance with FIG. 6. The left-hand section of FIG. 7 represents the actual measurement circuit, while the right-hand part represents the pump control system 8 and/or an evaluation circuit. FIG. 7 only shows evaluation for one acceleration sensor 19, but evaluation can also be provided correspondingly for the remaining acceleration sensors 20 and 21 in accordance with FIG. 6 or performed in a multiplex operation.

The measurement signal provided by the acceleration sensor 19, for example in the form of a voltage or charge, is continuously processes by the signal processor 26, for example a voltage or charge amplifier, and digitized by the A/D converter 27 at the frequency predetermined by the time control circuit 9. An event detector 6′, which in the simplest case is formed as a threshold value switch, monitors the digital signals provided by the A/D converter 27 and determines whether the registered acceleration values are within a permissible range which can be formed at least by an upper acceleration limit value but also by an acceleration band formed from a lower and an upper acceleration limit value. The converted signals are optionally provided to an external display means 25, an alarm means 12 and a data memory 10 in which the events are stored together with other variables, for example the time of the system clock 11. Alternatively, the event detector 6′ can of course also be arranged before the A/D converter 27 in the system flow.

Reference numerals 19, 26, 27 and 6′ together form a measurement circuit 24 and, in the case of a multiaxial design, are to be provided on each axis of the means. In order to read a number of acceleration sensors together, a multiplex method can be used such as will be known to the person skilled in the art. In accordance with a preferred embodiment, other components—such as the system clock 11, data memory 10, alarm means 12 and the external display means 25 —are only operated if the event detector 6′ triggers an alarm.

In principle, indicators and/or sensors which operate purely mechanically, for example the jolt indicator in accordance with FIG. 1, can be combined as desired with detector means which are to be read electronically or opto-electronically. This can cover the intended area of application whereby the means is kept ready at the manufacturer's, during distribution, transport, or when retained as a replacement means for another means, currently being used, without a power supply of its own. In such a case, the user can read off the indicator which operates purely mechanically and so be made aware of the danger of incorrect dosing or a malfunction. If the means is subsequently activated by being switched on, having an energy supply of its own inserted and the like, it can then additionally be controlled and evaluated electronically, as described above.

Critical acceleration values, for which the danger of damage to the mechanics and/or electronics of the means escalates when they are exceeded, are usually known from standard tests, for example a drop test. Aware of the mechanical and electrical configuration of the means, such acceleration limit values can also in principle be calculated or simulated or predetermined by safety regulations. In accordance with the invention, the acceleration limit values of the indicators and/or sensors mentioned above are preferably adapted to the acceleration limit values of the means (i.e., the devices, apparatus, etc. in accordance with the present invention, for example injection devices or infusion pumps). A number of sensors and/or indicators, having different acceleration limit values, can of course also be used.

If the means is electronically controlled and evaluated, an optical or acoustic warning indication can be given to the user in the usual way, which indicates that there is a danger of incorrectly dosing or malfunction and that it would seem advisable to have the means checked or to return it to a retailer or manufacturer. In response to such a warning indication, the user can then independently decide how to respond to this. Preferably, however, the injection and/or infusion means continues operating, even given the danger of a possible incorrect dosing, since it is a medical active agent or the like which is to be administered. However, providing it has been determined that the means has not yet been operated for the first time, a block on the means can be triggered. In principle, however, a block can also be triggered in all of the embodiments cited above, in particular those in which the means is evaluated electronically, when acceleration limit values are exceeded.

It should be understood that the source of accelerations or acceleration values may be external to the administration means, e.g., from shipping or handling, or “internal,” e.g., generated by or during the intended use, e.g., by the administering mechanism or dosing mechanism. Also, while the present invention has been described above in connection with detecting acceleration values as parameters for occurring mechanical stresses, the idea in principle is that other parameters which are significant for the operational reliability of the means can also be detected and evaluated by means of suitable sensors, for example temperatures, jumps in temperature, air humidity in the vicinity and/or in the interior of the casing of the means, the presence of liquid in the interior of the casing of the means and the like. Such forms of use are therefore to be regarded in principle as embodiments of the invention described above, as will be immediately clear to the person skilled in the art when studying the above description.

The present invention can embody and/or be used with a variety of administering or dispensing devices, including injection or infusion devices or apparatus such as are known from the prior art for administering medically active agents, or therapeutic or diagnostic agents into human, animal or vegetable tissue, in doses. A particularly preferred field of application is portable injection or infusion means for self-medication by patients. Examples of such means are injection pens, as known for example from DE 199 00 827 C1 belonging to the Applicant hereof, or portable infusion pumps for dispensing a number of comparatively small doses of a medicinally active agent over a comparatively long period of time. Such portable infusion pumps are known, for example, for dispensing insulin long-term in diabetic patients and can discharge the insulin via a 31 gauge needle.

The accompanying figures and this description depict and describe embodiments of a apparatus and methods in accordance with the present invention, and features, steps and components thereof. Although electronic, e.g., digital apparatus, components and methods are contemplated in some embodiments, the present invention is also intended to encompass “hard” or analog apparatus, components and methods.

Unless specifically disclosed or taught, any suitable coupling or linking methods and apparatus, including suitable mechanical or electronic components or devices, may be used in the present invention. For example, the present invention may incorporate appropriate microprocessors, integrated circuits, chips, memory structures, wireless links, data storage technology, etc.

Any control circuit, microprocessor or controller, or microprocessors, for the present invention can comprise any controller or microprocessor-based system, and more than one may be involved, including those comprising a suitable central processing unit and suitable peripheral devices. As one skilled in the art will recognize, various implementations of program logic are possible. The program logic could be either hardware, software, or a combination of both.

In the foregoing description, embodiments of the present invention, including preferred embodiments, have been presented for the purpose of illustration and description. They are not intended to be exhaustive or to limit the invention to the precise forms or steps disclosed. Obvious modifications or variations are possible in light of the above teachings. The embodiments were chosen and described to provide the best illustration of the principals of the invention and its practical application or use, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A device for administering an injectable product in doses, comprising a casing, a container for storing the injectable product and an administering mechanism for administering the product in doses comprising at least one detector means for detecting accelerations, wherein the at least one detector means is rigidly connected to the casing.

2. The device as set forth in claim 1, wherein the device is an infusion pump.

3. The device as set forth in claim 1, wherein the detector means changes a state when a detected acceleration exceeds a predetermined threshold value.

4. The device as set forth in claim 3, wherein the detector means comprises a glass capillary comprising a measurement area which changes from a first color to a second, different color when the detected acceleration exceeds the predetermined threshold value.

5. The device as set forth in claim 4, wherein the glass capillary is exchangeably inserted into a viewing window associated with the casing, such that the glass capillary is visible from outside the casing.

6. The device as set forth in claim 4, wherein the first and second colors comprise color values which are detected by means of a light detector array comprising a light-emitting element and a light-receiving element and which is controlled by a reading impulse of a control circuit.

7. The device as set forth in claim 6, wherein the reading impulse is generated by a pump control system, synchronously with the administering of the injectable product.

8. The device as set forth in claim 7, wherein the reading impulse is generated periodically.

9. The device as set forth in claim 3, wherein the detector means either reversibly or irreversibly changes a resistance value in a measurement area when the detected acceleration exceeds the predetermined threshold value.

10. The device as set forth in claim 9, wherein the detector means comprises a glass capillary with a thin wire which tears when the detected acceleration exceeds the predetermined threshold value.

11. The device as set forth in claim 9, wherein the detector means comprises a glass capillary with a liquid, which comprises at least two electrodes, wherein the liquid changes its resistance value between at least two electrodes in a measurement area when the detected acceleration exceeds the predetermined threshold value.

12. The device as set forth in claim 11, wherein the resistance value is detected by means of a resistance measurement circuit which is controlled by a reading impulse of a control circuit.

13. The device as set forth in claim 12, wherein the resistance measurement circuit is a Wheatstone bridge circuit.

14. The device as set forth in claim 12, wherein the reading impulse is generated by a pump control system, synchronously with the administering of the injectable product.

15. The device as set forth in claim 14, wherein the reading impulse is generated periodically.

16. The device as set forth in claim 3, further comprising a storage means for storing a time data value which indicates when the detector means changed its state.

17. The device as set forth in claim 3, wherein the control circuit controls an external display when the detected acceleration exceeds the predetermined threshold value.

18. The device as set forth in claim 1, wherein the detector means reversibly changes a state when a detected acceleration exceeds a predetermined threshold value.

19. The device as set forth in claim 18, wherein the detector means comprises an acceleration switch which reversibly changes an electrical switching state when the detected acceleration exceeds the predetermined threshold value.

20. The device as set forth in claim 19, wherein the acceleration switch is non-conductive in a normal state.

21. The device as set forth in claim 20, wherein the acceleration switch forms an input of a control and evaluation circuit which detects the changed switching state of the acceleration switch.

22. The device as set forth in claim 1, wherein the detector means either detects accelerations continuously or detects continuous values.

23. The device as set forth in claim 1, wherein the detector means comprises a piezo-electrical sensor with a subsequent charge amplifier.

24. The device as set forth in claim 3, further comprising a control and evaluation circuit provided with a comparator means in order to determine whether the detected acceleration exceeds the predetermined threshold value.

25. The device as set forth in claim 24, wherein the control and evaluation circuit comprises a storage means for storing a number of discrete data sets which each indicate that the detected acceleration in each case exceeds the predetermined threshold value.

26. The device as set forth in claim 25, wherein the evaluation circuit is configured to assign each data set at least one of a time value when the detected acceleration exceeds the predetermined threshold value, a period of time during which the detected acceleration exceeds the predetermined threshold value, and the value of the detected acceleration.

27. The device as set forth in claim 26, further comprising memory means for storing a number of data sets, wherein the data sets are ordered according to the value of the detected acceleration, and the data set with the smallest value for the detected acceleration in each case is replaced by a stored data set when the value for the detected acceleration is larger than the acceleration of the data set with the smallest value for the detected acceleration in each case, and wherein a data set with the currently detected acceleration is stored.

28. The device as set forth in claim 1, further comprising a display means for indicating a detected acceleration.

29. The device as set forth in claim 1, wherein the at least one detector means responds isotropically to accelerations.

30. The device as set forth in claim 1, wherein the at least one detector means responds direction-dependently to accelerations.

31. The device as set forth in claim 30, wherein two or three detector means are provided which respond to accelerations along respective mutually orthogonal directions.

32. The device as set forth in claim 30, wherein the at least one detector means is orientated parallel to a printed circuit board which has an associated control circuit for the device.

33. The device as set forth in claim 1, wherein the device is a portable infusion means for dispensing a medical active agent long-term in doses.

34. The device as set forth in claim 33, wherein the medical active agent is insulin.

35. The device as set forth in claim 33, wherein the product is dispensed through a 31 gauge needle.

36. A device for dispensing precise amounts of a substance comprising at least one detector operably coupled to the device and at least one indicator operably coupled to the device and to the detector, said detector for detecting accelerations, wherein the detector is at least one of triggered and read in one of a continuous or clocked manner and prompts the indicator to change when a selected acceleration is detected.

37. The device according to claim 36, wherein the change is either reversible or irreversible.

38. Means for administering an injectable product in doses, comprising a container for storing the injectable product and an administering mechanism for administering the product in doses, comprising at least one detector means for detecting accelerations, wherein each detector means is rigidly connected to a casing section of the means for administering.

39. A device for dispensing precise amounts of a substance, said device comprising a detector operably coupled to the device and an indicator operably coupled to the device and to the detector, said detector for detecting accelerations, wherein the detector is at least one of triggered and read in one of a continuous or clocked manner and prompts the indicator to change when a selected acceleration is detected.

40. The device as set forth in claim 39, wherein the indicator comprises a measurement area which changes from a first color to a second, different color when a detected acceleration exceeds the selected acceleration.

41. The device as set forth in claim 40, wherein the detector comprises a light detector array operably coupled to the measurement area and comprising a light-emitting element and a light-receiving element, said array controlled by a reading impulse of a control circuit associated with the device.

42. The device as set forth in claim 39, wherein the detector either reversibly or irreversibly prompts a change in a resistance value in a measurement area when a detected acceleration exceeds the selected acceleration.

43. The device as set forth in claim 42, wherein the detector comprises a thin wire which tears when the detected acceleration exceeds the selected acceleration.

44. The device as set forth in claim 39, wherein the detector comprises an acceleration switch which changes an electrical switching state when a detected acceleration exceeds the selected acceleration.

45. The device as set forth in claim 39, further comprising a control and evaluation circuit comprising a comparator for determining whether a detected acceleration exceeds the selected acceleration.

46. The device as set forth in claim 45, wherein the control and evaluation circuit comprises a storage means for storing a number of discrete data sets which each indicate that the detected acceleration in each case exceeds the selected acceleration.

47. The device as set forth in claim 46, wherein the control and evaluation circuit is configured to assign each data set at least one of a time value when the detected acceleration exceeds the selected acceleration, a period of time during which the detected acceleration exceeds the selected acceleration, and the value of the detected acceleration.

48. The device as set forth in claim 47, further comprising means for storing a number of data sets, wherein the data sets are ordered according to the magnitude of the detected acceleration, and the data set with the smallest magnitude for the detected acceleration in each case is replaced by a stored data set in which the magnitude for the detected acceleration is larger than the acceleration of the data set with the smallest magnitude for the detected acceleration in each case, and wherein a data set with a currently detected acceleration is stored.
